(19) United States
(12) Patent Application Publication
(10) Pub. No.: US 2026/0232376 A1
(43) Pub. Date: Aug. 13, 2026

(54) EASY LASER SYSTEM

(71) Applicant: GUANG777 LLC, New York, NY (US)

(72) Inventors: Gaung Yang, Blauvelt, NY (US); Jian Zhang, Blauvelt, NY (US); Vincent Tsang, Richmond Hill, NY (US); Xia Chong Li, Union City, NJ (US)

(73) Assignee: GUANG777 LLC, New York, NY (US)

(21) Appl. No.: 19/229,946

(22) Filed: Jun. 5, 2025

Related U.S. Application Data
(60) Provisional application No. 63/656,484, filed on Jun. 5, 2024.

(51) Int. Cl. A61B 18/20; A61B 18/00

(52) U.S. Cl. CPC A61B 18/20; A61B 2018/00476

(57) ABSTRACT

A medical laser hair removal treatment system and treatment method is provided that is adaptive to the patient's skin type, hair type and localized patient skin parameters. The laser hair removal treatment system and method includes using an application program that in operation receives and processes patient parameters, including the patient's skin type, hair type and localized patient skin parameters, and identification of the particular medical laser device to be used to treat the patient, and returns settings for the medical laser treatment device optimized for the particular medical laser treatment device and the patient's particular skin sensitivity to laser light at different body skin locations.

### BACKGROUND OF THE INVENTION

### 1. Field of the Invention

This invention relates to laser hair removal, broadly. More particularly, the invention relates to a medical laser hair removal treatment system adaptive to localized patient skin sensitivities, which system includes an application program that in operation receives and processes patient parameters, including localized skin sensitivities, and identification or specification of the medical laser treatment device to be used to treat the patient, and returns settings for the specified medical laser treatment device that will optimize the specified medical laser treatment device in view of the patient's particular skin sensitivities to laser light at the patient's different body skin locations under treatment.

### 2. Background of the Related Art

Conventional phototherapy systems are known. For example, WIPO published application WO2019/118773 (“the '773 WIPO Published Application”), which discloses a dynamic dosing system for phototherapy (which is a form of UV radiation therapy used for tanning) and associated devices, systems, and methods. The dynamic dosing system includes a self-service phototherapy kiosk (“SPK”) configured to emit UV radiation, a user interface coupled to the SPK, and a dosing system communicatively coupled to the user interface and the SPK. The dynamic dosing system determines initial, user-specific parameters to define a first individual phototherapy protocol, and adjustments to the initial, user-specific parameters based on user input related to any erythema response to a previous phototherapy treatment session. The SPK delivers UV radiation in accordance with the first individual phototherapy protocol and/or an adjusted, second phototherapy protocol taking into account the user's erythema response.

FIG. 1 of the WIPO '773 Published Application shows the first user interface 102a as comprising a touchscreen or monitor for entering and displaying run parameters and program information, which allows a user to control the SPK 104 during a phototherapy session. Information gathered from the patient user by any of the user interfaces 102 is stored locally, e.g., date of birth, gender, body weight, BMI, skin tone, Fitzpatrick skin type answers, hair color, eye color, number of freckles, propensity to sunburn, propensity to suntan, indication for use, and/or other parameters that may affect the UV treatment protocol. The dosing system 105 uses the gathered information to determine the user's skin type category, MED estimate, starting or initial dose, treatment frequency, dose increases, dose decreases, long-term treatment pattern, and/or other information relevant to phototherapy treatments and can make modifications to the current delivery dose (DDC) before treatment to account for photoadaptation, photosensitizing medication usage, recent UV exposure, and/or other adjustment parameters.

The dose modification routine 400 begins by receiving or determining an individual treatment dose (block 405). If a prior modification to the individual treatment dose has been stored in association with the user (decision block 410), the routine 400 moves to block 415 to equate the current delivery dose to the predetermined treatment dose. Once the current delivery dose has been set, the routine 400 moves to blocks 425, 430, and 435, where the SPK dosing system 105 modifies the current delivery dose based on information provided by the user regarding photoadaptation (block 425), medication (block 430), and/or recent UV exposure (block 435).

Summarizing, the phototherapy dynamic dosing system of the '773 WIPO Published Application is directed at UV tanning machines but does not teach or suggest a system for laser hair removal, including a server, a medical laser (hair removal) treatment device, and a web appIn operational at the server (and not at respective end user devices) that controls parameters for laser hair removal at different skin areas localized on a patient's body, which localized skin areas are subject to different sensitivities to laser treatment, where treatment reflecting the differing skin area sensitivities result in minimal discomfort and/or other ill effects to the patient at the localized skin areas.

US Published Patent Application US20200391049 (“the '049 US Published application”) discloses a dynamic dosing system for phototherapy that includes a self-service phototherapy kiosk (SPK) configured to emit UV radiation for tanning. The dynamic dosing system provides functions for user account management, skin type evaluation, treatment parameter determinations and adjustments, treatment blocking or warnings for some hazard prevention, treatment education and guidance, session records access, treatment regime determination, scheduling, and converting treatment parameter determinations into kiosk controls. Par 0016 of the '049 US Published application explains that the phototherapy system functions are performed based on user input, records of user data, guidelines and algorithms for treatment parameter selection, direct measurements, etc. These data sources can be accessed or implemented though one or more of: a phototherapy kiosk, a personal computing device, a server system, etc., to facilitate user control and user feedback for the phototherapy system.

Like the commonly-owned '773 WIPO Published Application, however, the phototherapy dynamic dosing system of the '049 Published Application is directed to UV tanning, and does not comprise a server, a medical laser (hair treatment) machine and a web-based application operational at the server (and not at respective end user devices) that takes in user data that is processed by the application to generate optimized settings at the different locations with different skin sensitivities, in view of uploaded patient localized skin sensitivity data, so the treatment technician or user can apply and calibrate the medical laser treatment device for the individual patient to control the medical laser machine in a way that is most effective and least offensive to the patient's skin.

US Published Patent Application US20180014777 (“the '777 US Published application”) discloses a method of dynamically adapting a facial treatment by a treatment applicator, based on a current facial skin profile. The treatments include treating the patient's skin, for example, to clean, moisturize, nurture, heat and cool the skin. The method includes using a sensor for measuring a current value of a variable skin characteristic of the facial skin of a patient, acquiring a personal skin characteristic of facial skin of the patient, calculating a current facial skin status of the patient according to the personal skin characteristic and the current value; determining instructions to operate a treatment applicator according to the current facial skin status; and instructing the treatment applicator according to the instructions.

Par. 0046 explains that the system/method improve performance of a computing unit operating the skin treatment applicator, by improving the selection of operation instructions to apply a facial treatment customized to the patients. Par 0059 explains that the skin treatment applicator 202 has sensors 214 designed to contact the face of the patient, and/or be positioned in proximity to the face without contacting the face, and which sensor 214 operate at visible light wavelength, at infrared wavelength, and/or other non-visible wavelengths, to perform direct measurements of, or generate data analyzed to obtain visual skin properties (e.g., color, shade, pigmentation, wrinkles, moles or others), physical skin properties (e.g., elasticity, thickness, topography, oiliness or others), and/or chemical skin properties (e.g., moisture content, skin elasticity detector, etc.), without contact of the skin.

Par 0063 explains that applicator element 216 may deliver energy to a facial segment, e.g., light, laser, RF, u/s, etc., whereinafter, pars. 0064-0067 teach that the laser therapy may be applied using any one or more wavelengths, such as a combination of 852 nanometer and 658 nanometer, for example at 20-100 milliwatt, that RF therapy can be applied at an intensity of 5-30 Watts and a frequency of 0.5-5 Megahertz (MHz), and that electrophoresis can be applied at a frequency of 30-200 Kilohertz (KHZ) and an intensity of 30-200 volt, for time periods such as between a few seconds and a few minutes, for example, up to about 10 minutes for each treatment type, or up to about 10 minutes altogether.

The system of the '777 US Published application calculates a current facial skin status of the patient in reliance upon code executing a machine learning algorithm trained using a training set storing at least one facial skin characteristic for each of a plurality of different patients. This includes at least one calculated current facial skin status of each of the plurality of different patients, based on a measured current value for a variable skin characteristic and a personal skin characteristic of each respective patient. Par 0006 explains that the method acquires an environmental condition characteristic, selected from: humidity forecast, temperature forecast, overcast forecast, pollution level, radiation level, and ultraviolet index forecast. But the '777 US Published application does not teach or suggest laser hair removal at many and varied places covering a person's entire body, still less in reliance upon a web-based application, operational at a server, and a medical laser (hair removal) machine, where the application optimizes laser hair removal machine parameters in reliance upon patient parameters designating a patient's localized skin sensitivities, etc., i.e., to produce a set of particularized medical laser device machine operating parameters such that the machine remove hair from various patient skin types (e.g., sensitivities) at various body locations, without or with minimal irritation of the skin thereat. Facial treatment is not hair removal.

U.S. Pat. No. 8,275,442 (“the '442 US patent”) discloses system and method of treatment planning for non- and minimally-invasive alteration of body adipose tissue for reduction and contouring of body fat. The system and method generate treatment plans by capturing current body part data (e.g., positioning, contour/shape, thickness of adipose tissue, etc.), determining desired outcome of treatment (e.g., percent reduction of adipose tissue thickness, degree of contour change, etc.), and determining treatment parameters to achieve desired results. Algorithms determine best-fit treatment parameters to use in treatment sessions.

System embodiment 400 includes a treatment plan generator 402 (which resides on a server 308), in communication with client computers, such as personal computer 410, workstation 412, laptop computer 414, etc. (“client computer”), via computer network 406. The treatment plan generator 402 communicates with a data storage device 408 (which operates as a repository for one or more databases 409), receives patient-specific data and other information relating to treatment plan requests, compares patient-specific data to the a priori and/or empirically-derived information stored and accessed from database(s) 409, calculates a best-fit combination of treatment parameters and formulates a treatment plan specific to a request to contour or sculp the patient's adipose tissue. The requests and/or treatment plan(s) are communicated through computer network 406 to/from one or more requesting client computers. The system and method of the '442 US patent enable medical practitioners to conduct remote physical examinations of a body target region, or image a target region using a remote medical imaging device, and then enter, download, or otherwise input data into a client computer for transmitting the data to the treatment plan generator 402.

The system and method of the '442 US patent, however, do not suggest using a medical laser (hair removal) system to remove hair from various skin types and body locations, nor a web-based application operating at a server that is provided with pertinent patient parameters that are processed to generate settings that optimize operation of the medical laser hair removal treatment device for the specific skin types, at the specific patient skin locations (for the specific patient), so that the treatment technician or other user can apply and calibrate the medical laser treatment device for the individual patient in view of that patient's skin sensitivities at the different skin locations.

### SUMMARY OF THE INVENTION

The present invention overcomes the shortcomings of the known arts, such as those mentioned above.

The inventive medical laser hair removal treatment system adaptive to localized patient skin sensitivities, sometimes shortened to “a medical laser hair removal treatment system,” is operated to treat persons using laser light to remove hair at different body skin locations, where the skin at the different body locations can and does exhibit varying skin sensitivities to the laser light treatment. The inventive medical laser hair removal treatment system processes data relating to the patient's skin type, hair type and the patient's skin sensitivities, at the different body locations and requirements of the medical laser treatment device that is part of the system, to generate and provide optimal settings for the medical laser treatment device that account for the patient's localized skin sensitivities and hair parameters during treatment, so the treatment technician or user can apply and calibrate the medical laser treatment device for the individual patient that attempts to accommodate the patient's skin and hair parameters at the locations to minimize the patient's comfortability during the treatment.

In one embodiment, the medical laser hair removal treatment system includes a medical laser treatment device and an application program, which application program may be referred to as “the web-based application.” The web-based application is operational at a server and provides a user interface (UI)/application programming interface (API), which receives patient parameters reflecting the patient's various localized skin sensitivities, hair conditions and the identity and/or specifications for the medical laser treatment device to be used. The web-based application processes the received and available data including the patient parameter data to generate optimized settings for driving the medical laser treatment device to optimally treat the patient and remove the patient's hair at the various skin locations. The “patient optimized” medical laser treatment device settings enable treating the patient's varying skin types, at the varying body locations without, or with minimal, irritations at the patient's various skin locations under treatment. The treatment technician or other user applies and calibrates the medical laser treatment device for the individual patient's skin sensitivities to laser light treatment.

In another embodiment, the medical laser hair removal treatment system includes a medical laser treatment device and an application program, sometimes referred to as a “plug-in” version of the inventive system. The plug-in embodiment is programmed to seamlessly connect to a compatible customer relation management (CRM) system through the API, resulting in a reduced time spent on record keeping on the client profile. The “plug-in” version is a web-based application that is operational at a server and provides a user interface (UI) and/or an application programming interface (API), whereby it can fetch user data from the compatible CRM system. Once a treatment technician selects the client he/she can input the patient parameters reflecting the patient's localized skin conditions and the identity for the medical laser treatment device, the web-based application will generate the optimized settings for driving the medical laser device according to the inventive principles. Once the treatment technician is finished with the treatment, he/she can input a pulse count of the medical laser treatment and click a button identified as “finish” on the UI and/or API. The web-based application will post service record data to the CRM system by UI/API, thereby creating an optimized treatment in view of each patient's skin sensitivities and reducing the time the technician would need to enter data into a separate record system for the treatment.

### BRIEF DESCRIPTION OF THE DRAWINGS

Further features and advantages of the invention will become apparent from the description of embodiments that follows, with reference to the attached figures, wherein:

FIG. 1 depicts an embodiment of the laser treatment hair removal system adaptive to localized patient skin sensitivities of the invention;

FIG. 2 depicts an alternative embodiment to the laser treatment hair removal system depicted in FIG. 1, utilizing an inventive web-based application;

FIG. 3 depicts a flow chart representing the process flow in a method of laser hair removal treatment adaptive to localized patient skin sensitivities utilizing the inventive web-based application;

FIG. 4A depicts a sign-in screen, presented to the treatment technician once he/she arrives at their company's link to the inventive web application;

FIG. 4B depicts a “start treatment” screen, presented to the treatment technician after the sign-in screen of FIG. 4A;

FIG. 5A depicts a display screen that requires the treatment technician to select the closest match to the patient's skin tone in the area being treated;

FIG. 5B depicts a display screen that requires the treatment technician to select the closest match to the patient's hair color in the area being treated;

FIG. 5C depicts a display screen that requires the treatment technician to select the closest match to the patient's reaction to sun;

FIG. 5D depicts a display screen that requires the treatment technician to select the closest match to the patient's hair thickness in the area being treated;

FIG. 5E depicts a display screen that requires the treatment technician to select the closest match to the patient's hair type;

FIG. 5F depicts a display screen that shows the localized skin parameter selected by the treatment technician, and the resulting settings that should be inputted into the machine, where the treatment technician can select whether the treatment id has been completed (X close button) or whether a “next area” is to be treated;

FIG. 6A depicts a display screen adapted to receive “optional” shade (pigmentation), coarseness and sensitivity settings;

FIG. 6B depicts a display screen adapted to highlight the parameters selected for hair color;

FIG. 7A depicts a display screen the “current day” clocked-in technicians, presented to a technician for choosing themselves if the technician is not signed into the device using the FIG. 2 plug-in system;

FIG. 7B depicts a display screen presented to the technician for choosing the patient, after sign-in, using the FIG. 2 plug-in system;

FIG. 7C depicts a display screen presented to the technician to choose the patient's body area to be treated, using the FIG. 2 plug-in system;

FIG. 7D depicts a display screen presented to the technician to choose the patient's skin tone of the treatment area, using the FIG. 2 plug-in system;

FIG. 7E depicts a display screen presented to the technician to choose the patient's hair color of the treatment area, using the FIG. 2 plug-in system;

FIG. 7F depicts a display screen presented to the technician to choose the patient's reaction to sun, using the FIG. 2 plug-in system;

FIG. 7G depicts a display screen presented to the technician to choose the patient's hair thickness at the treatment area, using the FIG. 2 plug-in system;

FIG. 7H depicts a display screen presented to the technician to choose the patient's hair type, using the FIG. 2 plug-in system;

FIG. 7I depicts a laser settings screen that the technician may need to change for the laser device before adjusting other settings based on the parameter selected in the screens shown in FIG. 7D to FIG. 7H;

FIG. 8A is a first portion of a flowchart depicting operation of the inventive system;

FIG. 8B is a second portion of the flowchart depicting operation of the inventive system; and

FIG. 8C is the last part of the flowchart depicting operation of the inventive system.

### DETAILED DESCRIPTION OF THE INVENTION

The following is a detailed description of exemplary embodiments of the invention depicted in the accompanying drawings. The exemplary embodiments are presented in such detail as to clearly describe the invention and are designed to enable a person of ordinary skill in the art to make and use the invention without undue experimentation.

The invention provides a medical laser hair removal treatment system adaptive to localized patient skin sensitivities, hair type, etc. and a medical laser hair removal treatment method that relies upon the system. The medical laser hair removal treatment system includes a medical laser treatment device and an application program, which may be referred to as the “web-based application,” operational at a server that provides a user interface (UI)/application programming interface (API) to receive patient parameters reflecting the patient's various localized skin sensitivities, hair type, etc. (see details below), the identity and/or specifications for the medical laser treatment device, and which returns optimized setting(s) for the medical laser treatment device to treat the particular skin locations in view of those location sensitivities, and remove hair of the patient's type with minimal skin irritations. The terms “client” and “patient,” and “user” and “technician,” may be used interchangeably in this description.

Specifically, the web-based application processes the received patient parameters reflecting the patient's various localized skin sensitivities, hair type or types at the skin locations under treatment and makes available data to generate optimized settings for driving the specified medical laser treatment device to treat the patient to remove hair at the skin locations under treatment. The “patient optimized” medical laser treatment device settings enable treating the patient's skin types and hair, at the varying body locations in a way that minimizes irritation, which can be a secondary effect of conventional treatments. The “patient optimized” medical laser treatment device settings are then preferably returned to the UI/API. A treatment technician or other user can apply and calibrate the medical laser treatment device with the optimized settings (that were returned to the UI/API) for the individual patient and the medical laser treatment device, in view of the patient's sensitivities to laser light treatment (i.e., the patient's particular hair and skin parameters).

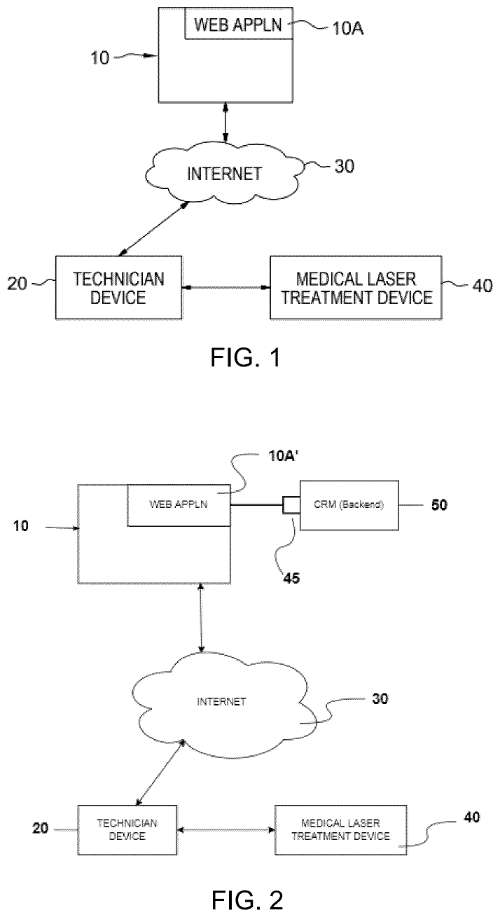

One exemplary embodiment of the medical laser hair removal treatment system, which is adaptive to localized patient skin sensitivities, hair type, etc. is shown in FIG. 1. The exemplary system includes an application program or web-based application (“web application”) 10A that operates on a server 10. The web application 10A provides a user interface UI/API programmed to receive or pull (when electronically connected to a web browser) particularized patient parameters (embodying the patient's medical data). Such pertinent patient medical data includes data associated with the patient's skin type, hair type, hair color, known sensitivities to sunlight (depending on various exposure conditions), corresponding body locations for hair removal (area), etc., and the identity and/or specification(s) for operating the medical laser treatment device 40, by which the particular patient is to be treated. Preferably, a treatment technician (or user) inputs this information (patient parameters) into the web application 10A using an electronic device 20, such as a desktop computer, laptop computer, iPad or other electronic tablet, smartphone, etc., connects to the UI/API over the Internet 30 using a browser in the device 20, and uploads/inputs the required data in screens presented by the UI/API by the web application (10A), which are configured to take in said data.

The web application 10A then processes the inputted patient parameters, etc., for a particular skin area or location, to generate the optimal medical laser treatment settings for treating said particular skin area(s) with the medical laser treatment device 40. The web application 10A takes into account the particularized patient parameters reflecting the localized skin sensitivities, and other patient parameters such as the hair type, color, etc. to optimize treatment at the skin area or location. In this way, the sensitivity settings for the medical laser treatment device are optimally adjusted in reliance upon the web application to accommodate the uniqueness of the different patient skin areas/types to be treated, and other patient characteristics or parameters that could affect treatment using the medical laser treatment device.

The optimal medical laser treatment device settings are then provided from the server 10/web application 10A to the treatment technician device 20, who then implements the displayed optimized setting(s) in the medical laser treatment device 40 and treats the skin location. Then, if there are further locations to treat, the treatment technician again uses the UI/API to the web application to input patient parameters relating to the “next” skin area for treatment to realize the optimal laser skin treatment setting for the same.

Additionally, the web application may be connected to an in-house CRM system 50 to further assist the treatment technicians' treatment workflow, as illustrated for an alternative embodiment of the inventive system depicted in FIG. 2. For example, the treatment technician with a tablet computer 20 can use the web application 10A′ operational therein to select the patient, who came for treatment, as well as the skin treatment area or areas for the patient. After that, the treatment technician will proceed to enter the patient's treatment area parameters, from which the web application 10A′ will then generate the optimal settings for the skin treatment area. In either case, the treatment technician, applies and calibrates the medical laser treatment device 40 for the individual patient in reliance upon the optimized settings to remove the patient's hair at the planned hair removal locations (i.e., skin treatment areas) with minimal discomfort and irritation. Once the treatment is implemented, the treatment technician can use the web application to save the treatment record into the CRM system.

The FIG. 2 medical laser hair removal treatment system may be referred to as a “plug-in” version of the FIG. 1 embodiment. In the FIG. 2 system, the web application 10A′ is connected to the in-house CRM system 50 by web application (plugin) 45 through the usage of the API. With the web application (plugin) 45, the web application 10A′ is able to retrieve data from the CRM 50, such as clocked in technicians, checked in patients for the day, and patient purchased sessions. Treatment technicians can then select the appropriate information for service record later. The following steps will be the same as FIG. 1, in which the technician will input the localized body area parameters into the web application 10A′. The server 10 takes the input parameters to generate the optimized settings for the laser treatment device 40, which gets displayed in technician device 20. Treatment technicians adjust the laser treatment device 40 with the returned optimized settings and process to perform treatment on the patient. Once the treatment is completed, the treatment technician can enter the pulse count of the treatment into the web application 10A′ and save to post the necessary data needed to construct a clear and concise service record to the CRM 50. This essentially removes the need for the treatment technician to enter the service record on another application.

The after-treatment service record is sent by the technician to the CRM database. Patient optimized settings are available in input device 50, and typically not sent directly to the medical laser treatment device 40. The benefit of having technician device 20 connected to device 40, is to allow communication between the two devices, minimizing bad laser settings from the technician and saving time required to input the settings. Please note that as used herein, a user is the one using input device 20 in FIG. 1 or device 50 in FIG. 2. It is generally the technician who is performing the treatment on the patient. However, if the client is using the input device in a patient self-treat situation, then the user would be the client. Please note that in-house CRM 50 (FIG. 2) may be thought of, or operated as a compatible laser hair removal treatment record system; compatible laser hair removal treatment record system, while not expressly shown, is present as a software plug-in.

The invention further provides a process or method for optimizing medical laser hair removal treatment system operation in view of both a patient's localized skin sensitivities to laser light exposure (and other patient parameters) and the actual settings protocol of the medical laser hair treatment device that controls and administers the medical laser hair removal treatment.

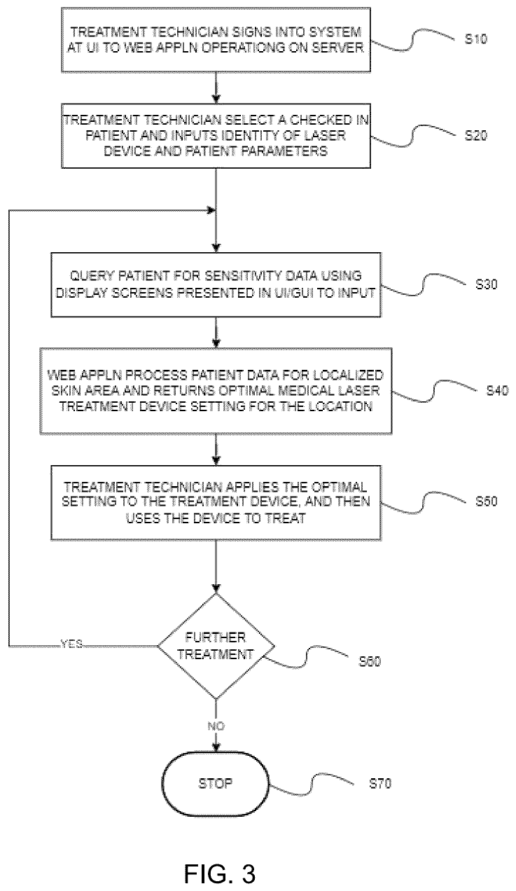

FIG. 3 is a flowchart setting out one embodiment of the inventive process, which relies on the medical laser hair removal systems illustrated in FIGS. 1 and 2. As may be seen in FIG. 3, a block S10 indicates a step by which the treatment technician signs into the system, via the UI/API provided by the web application (10A, 10A′) operating at the server 10, 10′. The standalone version is accessed via a link entered on the browser (see web page at “easylaser.co/u/SatoriLaser”), where the optimized parameters are loaded onto the web application 10A/10A′. The entered data are then displayed on the technician device 20 (see FIGS. 1 and 2). The link is generated and ready to use when the client registers and purchases the medical laser hair treatment service on the website, operated via server 10, or 10′/web application 10A, or 10A′.

The web application supports administrative accounts and can require (depending on configuration) 2-factor authentication, such as email identification and phone number, as known. The system supports employee accounts, which preferably similarly require two-factor authentication. Also, a location IP Address, while optional, is preferred for added security on top of the 2-factor authentication. This would require a business name & service address.

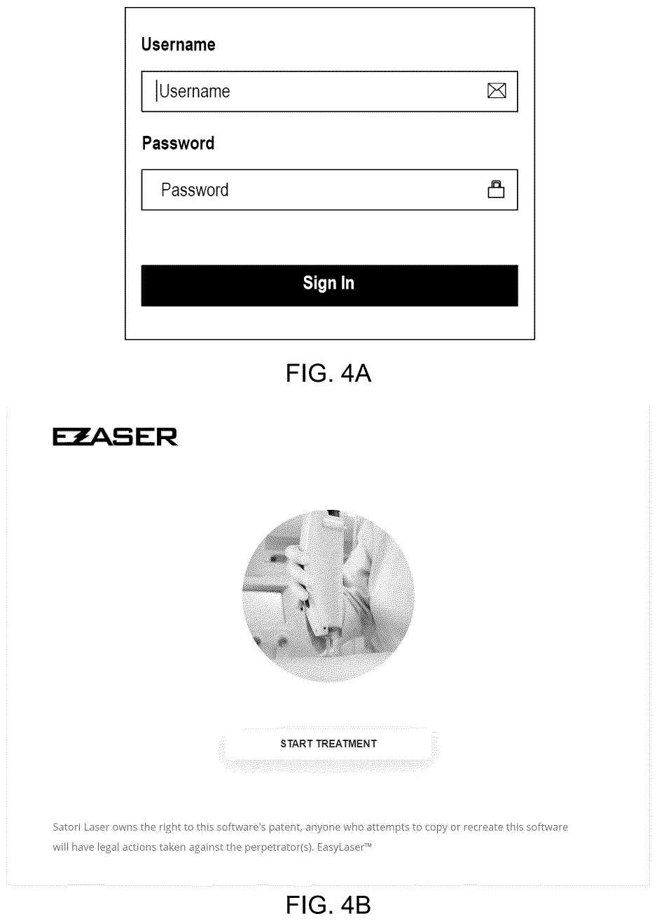

Block S20 (FIG. 3) represents a step by which a treatment technician, or other medical professional, logs in at the web pages presented at the user interface (UI)/API, provided by the server 10, 10′) for a particular patient, including patient identification information. To that end, FIG. 4A depicts a sign-in screen presented to the treatment technician once he/she arrives at their company's link and prompted to sign into their account (whether relying upon the FIG. 1 or 2 system embodiments). FIG. 4B depicts a “start treatment” screen. Preferably, the treatment technician also identifies the medical laser treatment device, by choosing from a list of known medical laser treatment devices. The patient parameters in the aggregate reflect the patient's sensitivities, or potential sensitivities, to laser hair treatment. The treatment technician then (or previously) queries the patient for the medical data “requested” by the display screens presented by the UI/API, as indicated by block S30. Alternatively, the treatment technician may carefully observe the patient, and respond to the data requests provided by the UI/API operated by web application 10A.

Please note that the web application 10A or 10A′ at the servers 10 or 10′, present the UI/API to the technician browser. Such web application is a CRM system that contains patient's information, scheduling data, transaction history, etc. The laser settings are displayed after retrieving the settings from technician device 20 (FIG. 1) to be provided to medical laser treatment device 40′ (FIG. 2) are saved to the database in server 10/10′ for use by the web application 10A, 10A′. The CRM 50 and plug-in 45 may be part of the web application operational at servers.

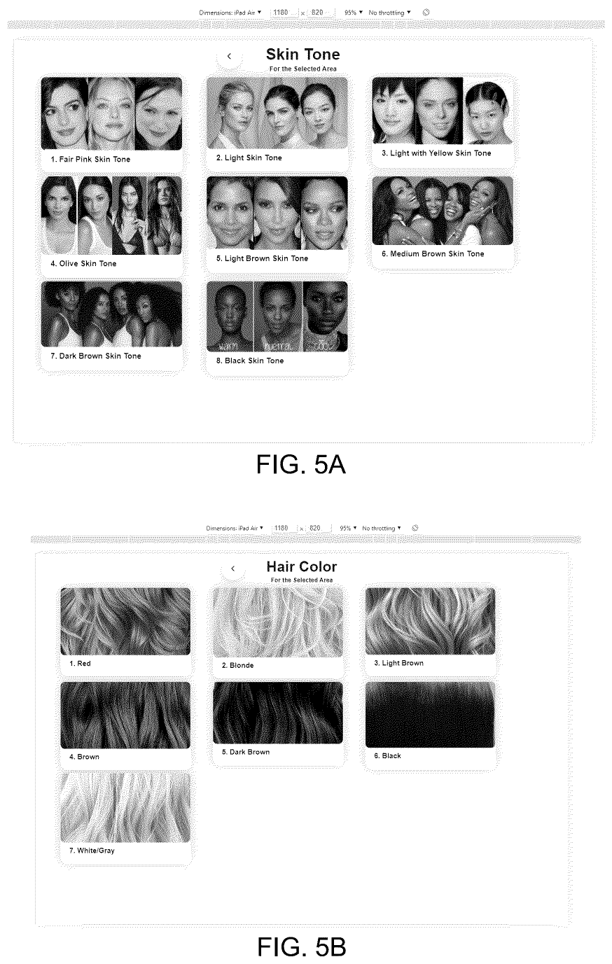

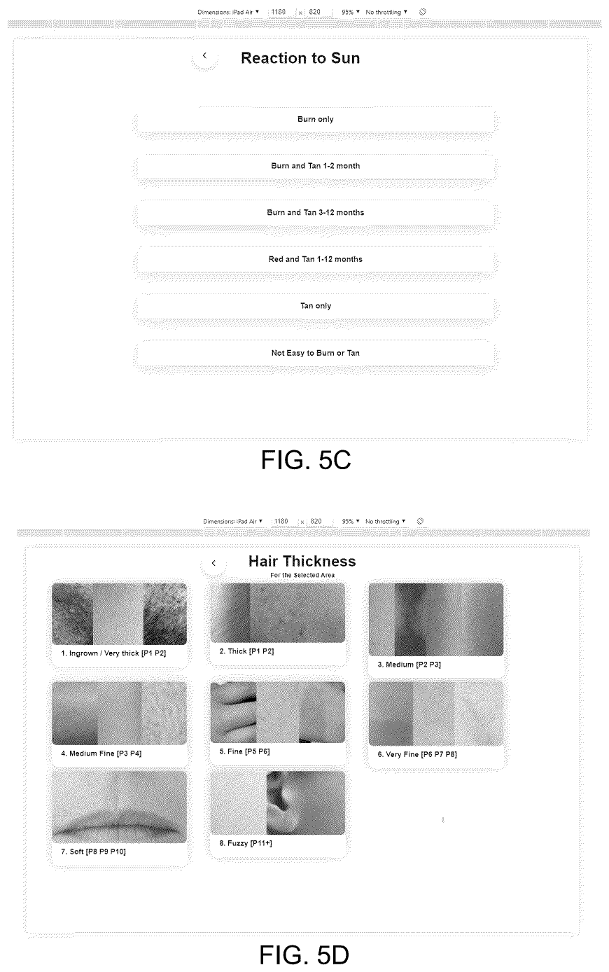

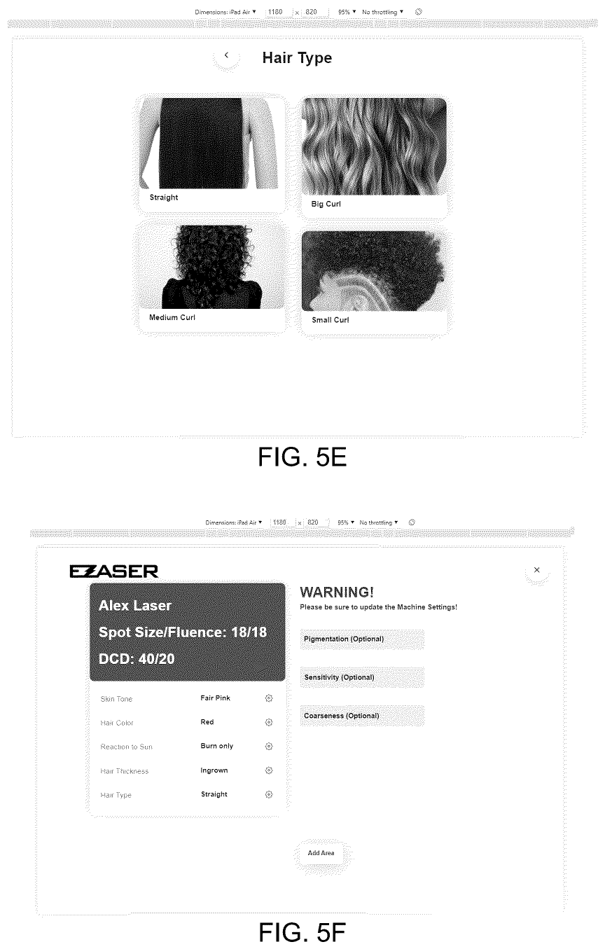

FIG. 5A presents a display screen adapted to receive input on the patient's skin tone in the area being instantly treated. FIG. 5B presents a screenshot that requires the treatment technician to select the closest match of the patient's hair color. FIG. 5C presents a screenshot that requires the treatment technician to select the closest match of the patient's reaction to sun; this requires the treatment technician to query the patient. FIG. 5D presents a screenshot that asks the treatment technician to select the hair thickness in the localized treatment area, where FIG. 5E is presented to the treatment technician to identify the actual patient's hair type. Once the 5 conditions are selected, the system (i.e., the web application) processes the data requested to treat the patient for a particular skin location. FIG. 5F depicts an exemplary screen shot of the optimal laser setting for the 5 conditions that will be displayed on a “settings” page (preferably).

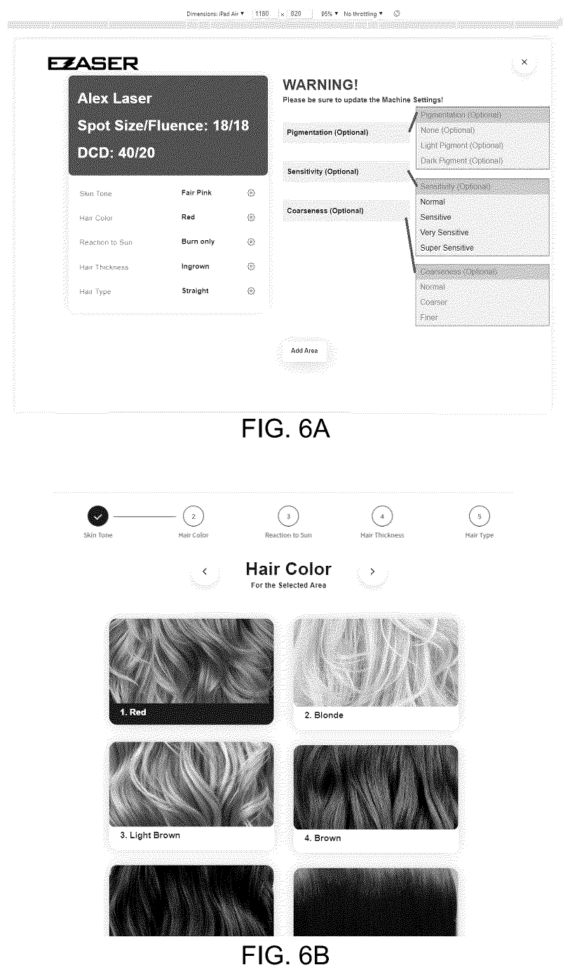

Optional settings, Shade, Coarseness and Sensitivity, are depicted in FIG. 6A, and are dependent upon the client's conditions and/or sensitivities. If the localized area under treatment is observed by the treatment technician to have different shades of skin color, or is sensitive, the treatment technician may adjust the shade or sensitivity setting to accommodate the uniqueness of that area which will automatically adjust the settings to achieve optimal results for the area.

Block S40 represents the step by which the web applications process the uploaded patient sensitivity data and returns an optimal laser setting for treating the patient at the location with the medical laser treatment device 40. This information enables the treatment technician to start treating the patient. The treatment technician then configures or adjusts the medical laser treatment device to treat the particular localized skin area, as represented by block S50.

For that matter, patients may need more than one area treated. Decision diamond S60 (FIG. 3) represents a step in the process where the treatment technician determines if there are further areas to treat, and if so, returns to block S30. In that case, the treatment technician selects Next Area which will return the interface to the Skin Tone selection with the previous settings highlighted in a black background allowing technicians to easily see what was selected for their previous area (see, for example, FIG. 6B). If not, the process ends (S70).

Please note that highlighting using a black background is not meant to be a limitation, the border could be highlighted with brightness, boldness, underlining, etc. The technician will repeat the same process as before for the client's next area, as represented by decision diamond S60. Preferably, when the technician is done with treatment of the client/patient, the technician will select Finish at the laser settings screen, which will reset the system and prepare the technician for the next client.

The step represented by Block 50 (FIG. 3) could alternatively be implemented directly. That is, the web application 10A/10A′ could send the calculated optimized setting(s) for a particular skin location directly to the medical laser treatment device 40′ (FIG. 2), as explained above, obviating a need for the treatment technician to receive the optimized setting data in a browser window on technician device 20, and then configure In an embodiment, the medical laser treatment system relies upon Candela's GentleMAX Pro medical laser treatment device. As known to the persons skilled in this art, however, just about any available medical laser hair removal treatment device may be used, according to the inventive principles. For that matter, and as also known to the skilled persons, optimal settings for other medical laser treatment devices (i.e., other than the Candela GentleMAX Pro medical laser treatment device) are readily calculated according to the inventive process, for example, in reliance upon an energy to fluence conversion factor Table, such as the Table 1, reproduced as follows:

TABLE 1
Energy to Fluence Conversion Factors

| | |
|---|---|
| Spot Size | Fluence Conversion Factor |

| | | |
|---|---|---|
| 1.5 | mm | 59.590 × External Meter Energy |
| 3 | mm | 14.150 × External Meter Energy |
| 3 × 10 | mm | 4.250 × External Meter Energy |
| 5 | mm | 5.100 × External Meter Energy |
| 6 | mm | 3.540 × External Meter Energy |
| 8 | mm | 1.990 × External Meter Energy |
| 10 | mm | 1.270 × External Meter Energy |
| 12 | mm | 0.880 × External Meter Energy |
| 15 | mm | 0.566 × External Meter Energy |
| 18 | mm | 0.393 × External Meter Energy |
| 20 | mm | 0.318 × External Meter Energy |
| 22 | mm | 0.263 × External Meter Energy |
| 24 | mm | 0.221 × External Meter Energy |

As mentioned above, the plug-in version 45 is accessed via a web application 10A/10A′ is stored in a memory in the CRM 50, i.e., the medical treatment laser system. Treatment technicians must sign into their CRM 50 platform to access the web application (for example, the “EASY LASER™ web application), which is IP address locked. Having the IP address locked adds a layer of security to ensure only authorized users are allowed to use the system by whitelisting their IP. Users of the location, upon registration will supply the server 10 (FIGS. 1 and 2) with their IP address, which will whitelist any users connecting to the web application 10A. Anyone not connecting from the location's IP address, will have their connections rejected.

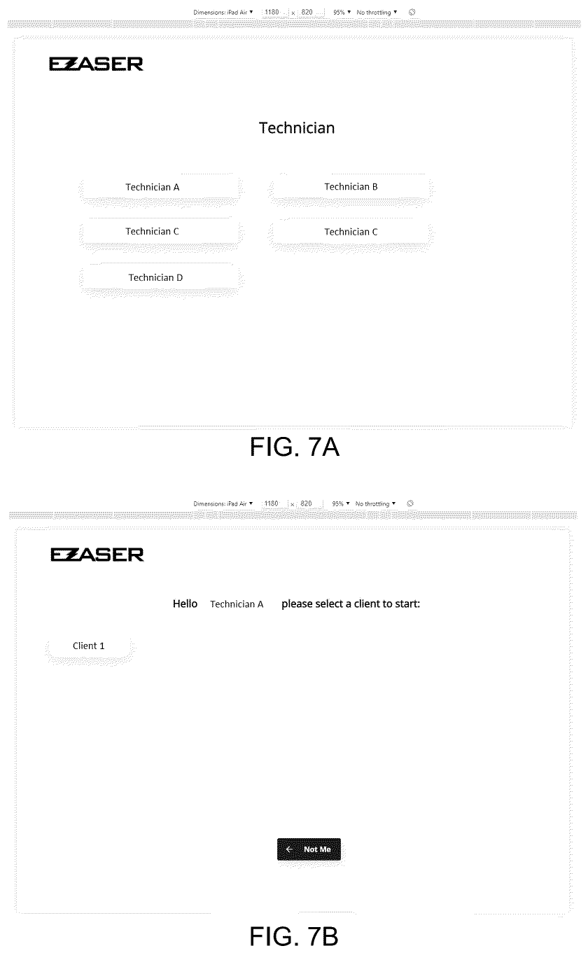

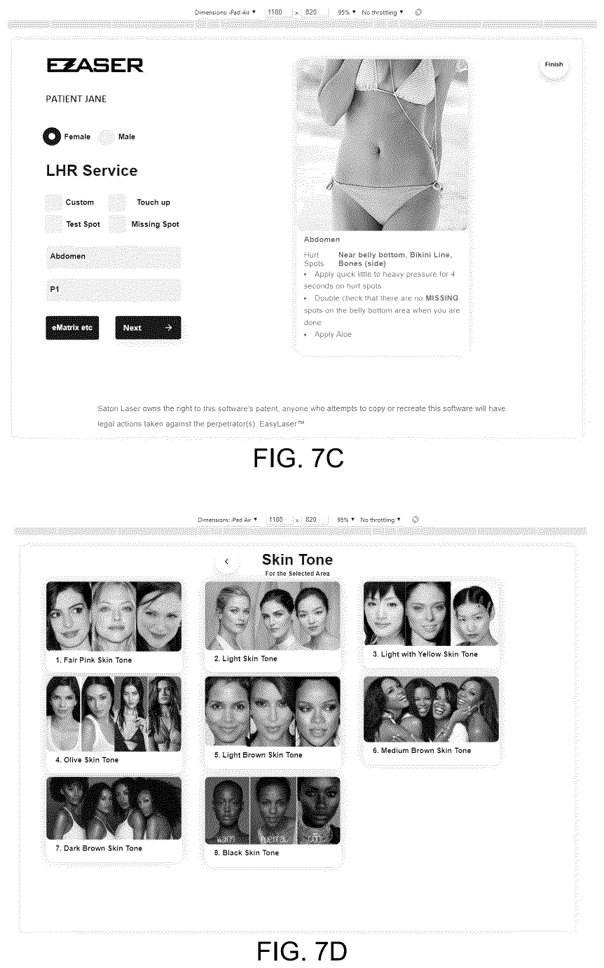

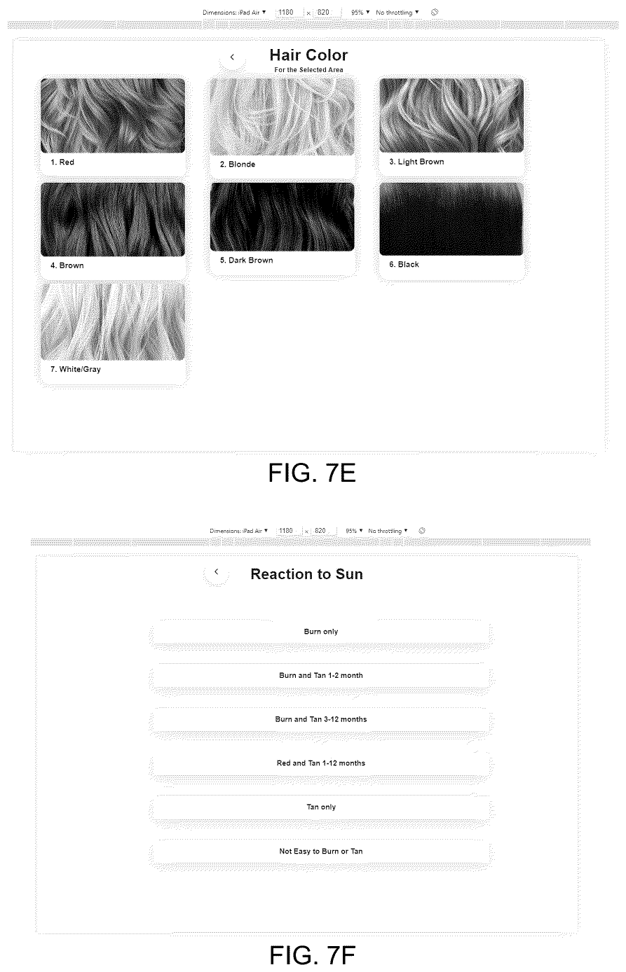

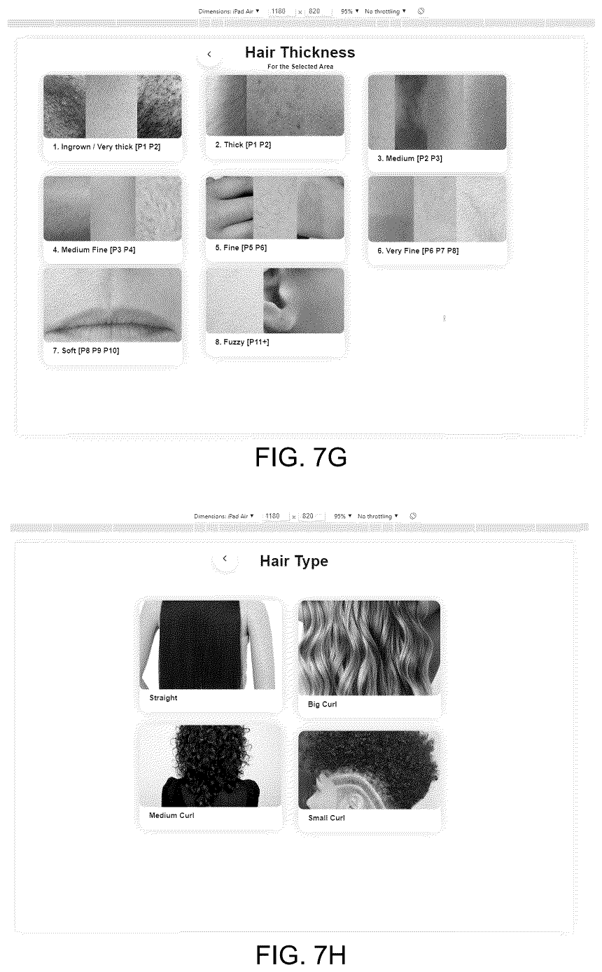

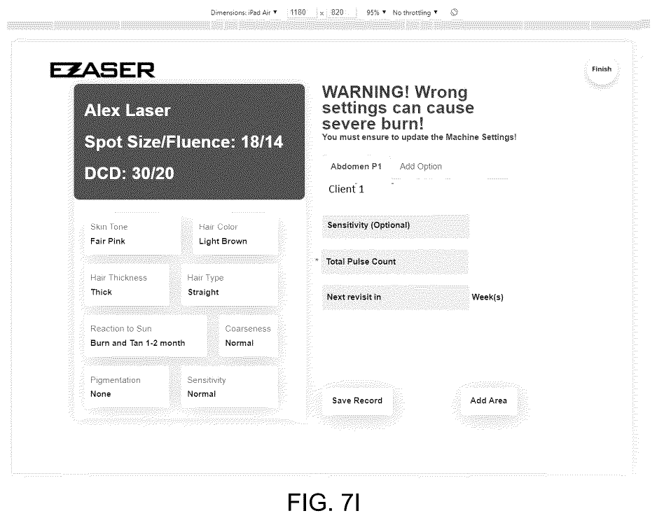

When the treatment technician actuates the plug-in 45, they are presented (via the display screen) with a welcome page depicted in FIG. 7A. Preferably, a list of names is presented to the treatment technician, from which the technician can pick their name. The chosen name is automatically populated by the technician's clocking into a timecard system that is part of the CRM the store, to start work for the current day. Although this seems to be an additional step, it saves users the task of signing in and out of the system, which takes much longer and is less user friendly. This often happens when multiple technician users share one device. The treatment technician and treatment area are both “selected.” However, if treatment technician has a logged in session, then there is no need to select treatment technician, as the web application will auto select the logged in technician.

Once the treatment technician has selected his/her own name in reliance upon the screen in FIG. 7A, she/he will have to select a client from the client list. The client list is connected to the check-in plugin of the backend where users check into the store they're in. So, the client names are automatically populated based on the clients checked-in to the store for the current day. FIG. 7B presents an exemplary screen for doing so. The treatment technician then selects the body area and the session number for the patient being treated (see FIG. 7C). Selecting the body area will cause the web application 10A, 10A′ to present screens that show the treatment technician technique and tips for servicing that area. The technician then selects the patient's skin tone (FIG. 7D), hair color (FIG. 7E), reaction to sun (FIG. 7F), hair thickness (FIG. 7G) and hair type (FIG. 7h). Once the 5 conditions are selected, the optimal laser setting is calculated and displayed on the settings page (FIG. 7I), allowing the technician to adjust the laser machine to these settings to start treating the client. The optional settings Shade and Sensitivity shown in FIG. 7J depend on the patient's conditions. If the area under treatment has different shades of skin color, or is sensitive, the technician may adjust the shade or sensitivity setting to accommodate the uniqueness of that area which will automatically adjust the settings to achieve optimal results for the area.

The inventive system and method also provide for a pulse count for use to record the number of pulses used on the selected area, localized skin areas. The next revisit (weeks) is the number of weeks recommended by the technician the next time this specific client should come for their next treatment. And a save button saves the settings into the patient's profile in the backend system. If the treatment technician presses “Next Area” or “Finish,” without saving first, an alert will be prompt if technician wants to exit page without saving.

Please note that the CRM 50 is a “compatible laser hair removal treatment record system.” And a general difference between the standalone (FIG. 1) and plugin (FIG. 2) systems is that the plugin system (with API plugin 45) is connected to its CRM 50 to store client's data when treatments are done while the standalone system (with medical treatment device 40) can be used by itself without storage of client treatment data. Please further note the technician device 20 and medical treatment device 40 in FIG. 1 (standalone) are connected by the user/technician's manual operation. The user/technician inputs the localized skin parameters into technician device 20, which are then processed to generate and output the optimized settings. The user/technician then configures the medical laser treatment device 40 based on the output of technician device 20.

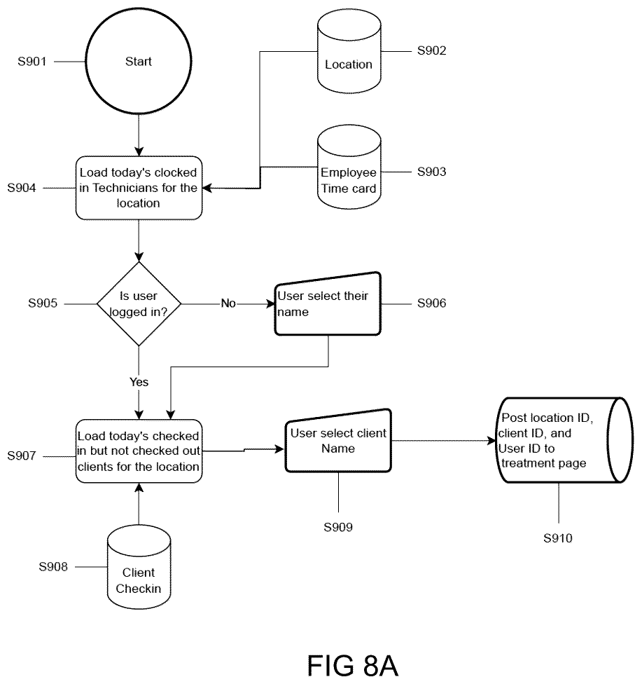

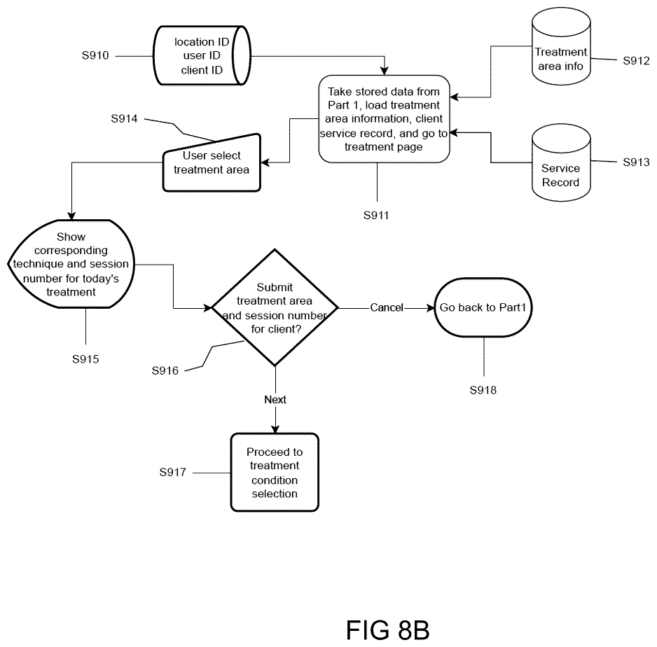

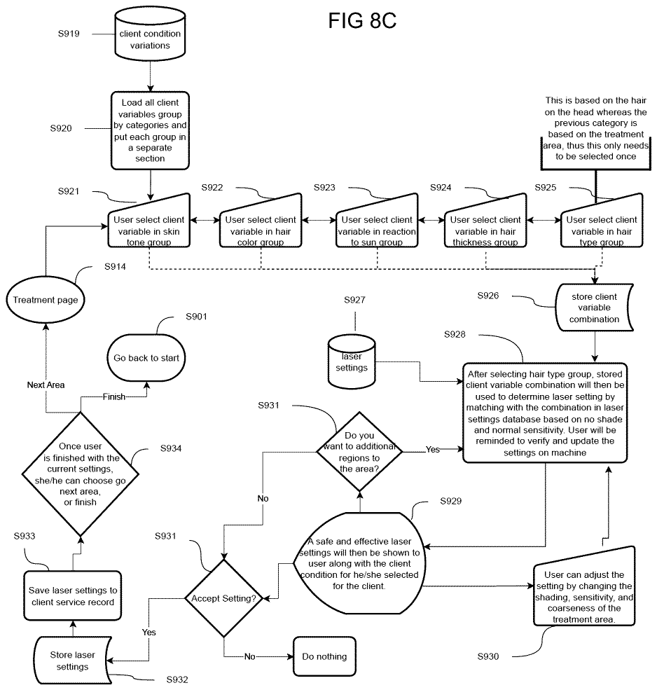

FIGS. 8A, 8B and 8C present a flow chart of another embodiment of the FIG. 2 medical laser hair removal treatment system adaptive to localized patient skin sensitivities operation, in first, second and third parts, respectively. In FIG. 8A (“the first part”), step S901 indicates start of the web application 10A (of FIG. 1), or 10A′ (of FIG. 2) where step S904 indicates that the day's (“today's”) “clocked in” or “logged in” technicians for the location, e.g., office in which the medical hair removal procedure is implemented in reliance upon the inventive system. For example, step S904 might rely upon input of a technician (employee) timecard (S903), at the location (which location is input as indicated by step S902, as shown. Preferably, the timecard data is retrieved from the CRM by the API. Step S905 represents (by the decision diamond) a decision that is made as to whether the user is logged in. If the decision is “no,” the user selects his/her name from a list presented in a screenshot (step S906), which is direct client manual input. But if “yes,” then the days' “checked in but not checked out” clients for the location are presented. The user selects the right name in step S909 (which is direct user manual input) and the location ID, client ID and user ID are posted to the treatment page in step S910. Step S908 indicates that client check-in data is stored in and accessible from a database, or simple memory.

The algorithm or process continues, as shown in FIG. 8B, where step S911 represents taking stored data from the first part (FIG. 8A), load treatment area information, client service record data and move on to the treatment page. Step S910 indicates the storage device providing the location ID, user ID and client ID, where step S912 represents the stored treatment area information and step S913 represents the service record. In step 914, the technician/user selects the treatment area and in step S915, the corresponding technique and session number for “today's” treatment. Thereafter, a decision must be made (see the decision diamond represented as step S916) whether to submit a treatments area and session number for the client. If yes, the process moves on to step S917, proceeding with the treatments condition selection (see part 2; FIG. 9C) and if not, the process returns to the process portion represented by FIG. 8A (part 1), whether step S904 or S905.

The algorithm or process continues, as shown in FIG. 9C. Step S19 represents a store of client condition variations that are communicated for use in step S920. Step S920 represents a step by which all client variables groups by category and then putting each group in a separate storage section or location. The process then moves on the step S921, whereby the user manually selects the client's variable in the skin tone group, step S922 where the user manually select's the client's variable in the hair color group, step S923, where the user manually selects the client variable in reaction to sun group, step S924, where the user manually selects the client variable in the hair thickness group and step S925, where the user manually selects the client variable in the hair type group. After processing any or all of steps S921, S922, S923, S924 and S925, the process stores the client variable combination, as represented by step S926.

Step S928 represents a step occurring after the user technician has selected the hair group type, stored client variable combination will then be used to determine laser setting by matching with the combination in laser settings database based on no shade and normal sensitivity. Step S928 includes that the user will be reminded to verify and update the settings on the machine, i.e., the medical laser treatment device. Step S927 indicates that the stored laser device settings are provided for reliance upon step S928. Step 929 represents a step of showing the safe and effective laser settings to the user technician along with the client conditions that the user/technician selected for the client. The process then determines whether to accept the setting, as represented by decision diamond step S930. If not, the process moves to step S931, where the technician user can adjust the setting by changing the shading, sensitivity and coarseness of the treatment area at a specified region and moves back to Step S928.

If the setting (S930) is accepted, the process moves on to step S932, where stored data such as the laser settings are then saved to the client service record in step S9333. The process then determines whether there is a next area of the client for treatment, or alternatively, the client wishes to finish, as represented in the decision diamond step S934. If there is a “next area,” the process moves step S914 (part 2, FIG. 8B), where the client user selects the treatment page. If the client technician's treatment of the patient/user is finished, the process moves back to the start step S901 (part 1; FIG. 8A)

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit the scope of the invention, except as set forth in the following claims.

We claim:

1. A laser hair removal treatment system adaptive to localized patient skin and hair parameters, comprising:
a medical laser treatment device; and
an application program operational to receive and process patient parameters reflecting the patient's hair type, skin type and localized skin sensitivity of body skin locations of the patient for treatment and return a setting for the medical laser treatment device optimized for the particular medical laser treatment device, and for the patient's particular skin sensitivity to laser light at the body skin locations for treatment.

2. The laser hair removal treatment system of claim 1, wherein the application program is programmed to enable a treatment technician to login, identify a patient to be treated, input patient parameters reflecting the patient's sensitivity to laser hair removal, and return a medical laser treatment device setting optimized for the patient in view of the patient's sensitivity and limitations of the medical laser treatment device.

3. The laser hair removal treatment system of claim 1, further comprising a server, wherein the application program is programmed to operate at the server.

4. The laser hair removal treatment system of claim 3, wherein the application program is programmed to communicate the returned setting to the treatment technician, enabling the treatment technician to manually enter the returned setting into the medical laser treatment device.

5. The laser hair removal treatment system of claim 4, wherein the treatment technician communicates with the server and application program over a network using an electronic device with a browser, and the application program returns the optimized setting to the browser.

6. The laser hair removal treatment system of claim 4, wherein the treatment technician communicates with the server over a network using an electronic device with a browser, the server returns the optimized setting directly to the application program over the network and the treatment technician manually inputs it into the medical treatment device.

7. The laser hair removal treatment device of claim 1, wherein the optimized settings may be returned to the treatment technician for the skin location being treated via a display screen at a technician device or at the medical laser treatment device.

8. A method of laser hair removal treatment adaptive to localized patient skin and hair parameters, comprising the steps of:
providing an application program to receive and process patient parameters reflecting the patient's hair type, skin type and localized skin sensitivity to laser hair treatment at a body skin location, and return a setting for a medical laser treatment device optimized for the patient's localized skin sensitivity to laser hair treatment at the body skin location;
a user uploading or inputting the patient parameters reflecting localized skin sensitivities for the patient to the application program; and
the application program processing the patient parameters reflecting localized skin sensitivities and returning an optimized setting for the medical laser treatment device for the skin locations defined by the patient parameters.

9. The method of claim 8, wherein the application program is operational at a server, and provides a user interface (UI) or application programming interface (API) that receives the patient parameters reflecting the localized skin sensitivities and provides the returned optimized setting for the medical laser treatment device.

10. The method of claim 9, wherein the user relies upon an electronic device to communicate with the application program.

11. The method of claim 10, wherein the application program provides the returned setting to a browser in the treatment technician electronic device.

12. The method of claim 11, wherein the user enters the returned setting into the medical laser treatment device.

13. The method of claim 8, wherein the patient parameters include any patient medical data, and data associated with the patient's skin type, hair type, hair color, known sensitivities to sunlight corresponding body locations for hair removal.

14. The method of claim 8, wherein the user is a treatment technician.

15. The method of claim 8, wherein the user is the patient.

16. A computer program product comprising program code means embodied in a non-transitory computer readable medium, which upon processing by a computer operating as a server, executes the program code means to implement the method of claim 1.
